(19) United States
(12) Patent Application Publication
(10) Pub. No.: US 2023/0226267 A1
(43) Pub. Date: Jul. 20, 2023

(54) DOME CATHETER SYSTEM FOR TRANSCUTANEOUS VACUUM ASSISTED CLOSURE OF PERIANAL OR ENTEROCUTANEOUS ABCESSES AND FISTULA TRACTS AND METHOD THEREOF

(71) Applicant: Semiflex Dome System Sp. Z.o.o., Poznan (PL)

(72) Inventor: Slawa Helena Madelska, Poznan (PL)

(21) Appl. No.: 17/578,816

(22) Filed: Jan. 19, 2022

(51) Int. Cl. A61M 1/00; A61M 25/00; A61M 25/01

(52) U.S. Cl. CPC A61M 1/90; A61M 25/0068; A61M 25/0108; A61M 2210/1067; A61M 2205/32

(57) ABSTRACT

A catheter for transcutaneous and transluminal vacuum closure of perianal or entero-cutaneous fistulas and abscesses containing a body ending in a dome having a low-friction surface and equipped with a series of openings on the surface that connect inside the catheter body into a catheter chamber, which is then connected to a tube connected to a vacuum generating system characterized in that the catheter body on the outside has edges arranged in the shape of many helicoid coils, and in the grooves between these coils the series of openings are made into the chamber inside the catheter.

### CROSS-REFERENCE TO RELATED APPLICATIONS

There are no cross-related applications.

### FIELD OF THE INVENTION

The present invention generally relates to a dome catheter system and method of installation thereof. In particular, the present invention relates to a dome catheter system comprising catheters having helical ridges and a lattice internal structure.

### BACKGROUND OF THE INVENTION

Perianal fistula in Crohn's disease (CD) affects almost one third of patients, greatly compromising their quality of life. This condition remains a very challenging problem to overcome both for physicians as well as surgeons, Despite new medical and surgical developments, the fistulas are only cured in less than one out of five patients after extensive medical and surgical management, affecting profoundly the socioeconomic situation and quality of life of the patient. In addition, perianal Crohn's fistulas are responsible for an enormous healthcare consumption. Management of perianal fistulas in Crohn's disease must therefore be considered as one of the most important unmet needs in patients suffering from inflammatory bowel disease (IBD). This has recently been recognized and highlighted by the European Crohn's and Colitis Organisation in the fifth scientific workshop addressing perianal fistula in Crohn's disease and by the Crohn's and Colitis Foundation (CCF) facilitating a working group brainstorming on “challenges in IBD research”.

Evidence is accumulating that medical therapy such as anti-TNF can only provide sustained clinical remission in less than one out of five patients compared to placebo. However, the data of the CCF supported PISA II trial (PISA Study: Multimodal treatment of Perianal fistulas in Crohn's disease: Seton versus anti-TNF versus Advancement Plasty) demonstrates that in the anti-TNF group, very few of the clinically externally closed fistulas show true healing on MRL A drop of the through levels is associated with the reopening of the external openings indicating that a clinical response, if any, is not durable. Overall, almost no MRI closure in the anti-TNF patient group and withdrawal of costly medication results in re-opening of the fistula (only 3% of patients on anti-TNF showed closure with MRI, as shown in the PISA Study II) while surgical closure was effective in nearly one out of two (44% of the patients who underwent surgical closure).

A primary beneficiary to a solution to the above stated problem are Crohn's Disease and Ulcerative Colitis patients which, according to the Centres for Disease Control and Prevention, includes at least 5 million people in Europe and North America with an additional 100,000 to 200,000 newly diagnosed cases each year in these areas alone. Moreover, annual direct healthcare costs of CD in Europe alone are estimated at approximately 4.6 to 5.6 billion Euros. Non-IBD patient groups are the secondary beneficiary to the above stated problem who may suffer from cryptoglandular perianal fistula, with the estimated incidence in Europe being 1.2-2.8 per 10 000 population, with a peak incidence between the age of 20 and 40 years.

A third category of patients benefiting from a solution to the above stated problem are patients which develop enterocutaneous abscesses and fistula as result of a surgical intervention of the digestive tract.

Current deep wound drainage systems are intended for passive drainage of fluids having a low viscosity such as blood and seroma following trauma or surgery. These are not well adapted for eliminated granular tissue as can be found in fistulas nor have the adequate rigidity of features to be inserted or removed through a perianal or enterocutaneous fistula.

Several systems for the management of deep abscesses and fistula tracts are known in the prior art, however they are almost exclusively passive draining systems operating on gravity.

Exceptionally, the Endo-SPONGE™ (B. Braun Medical, Melsungen, Germany), is a commercially available active device which has been used to address the unmet medical need of the holistic approach to fistula management per se via a natural orifice. It is designed for transluminal management of anastomotic leaks of esophageal or colorectal anastomosis and approved for the treatment of abscess cavities from anastomotic leakages of colorectal/coloanal and gastroesophageal anastomosis but is also used off-label in the treatment of fistulas and Problems related with existing solutions, local anesthesia, etc.).

Despite being adapted for use in perianal fistula, the Endo-SPONGE™ presents a great number of shortcomings.

Firstly, the use of the EndoSPONGE™ without transluminal access is painful to the patient for both insertion and removal. General anesthesia and hospital care are therefore typically needed. Moreover, the granulation tissues adhere to the sponge causing tearing and bleeding of the tissue when removing the sponge.

The Endo-SPONGE™ is further only supplied in one size and it is subsequently intended to be adjusted in size by a surgeon, therefore relying heavily on the skills of the specific surgeon. In the same vein, customizing the sponge increases the risk of sponge debris being left inside the wound.

Another disadvantage lies in the sponge not being visible in MRI scans, and therefore not allowing to specify the pace of removing it based on the state of healing seen in MRI scans.

Moreover, the organic debris passing through the sponge (such as blood or pus) causes the sponge to clog which compromises its function. According to B. Braun Medical, the sponge may stay in-dwelling for up to 3-4 days, but practical usage indicates that it may clog faster than indicated.

Finally, the granulation tissue may adhere to the sponge which causes tearing and bleeding of the tissue when removing the sponge.

Alternatively, surgeons also use hand-made catheter tips in fistula management. These are catheter tips tailored to the patient's specific situation and are typically made using punctured drain ends hand-braided with silver mesh. The disadvantages of this solution include the inability to standardize and certify this method, risk of faulty execution of the method, and the lack of visibility of the drain tip during MRI imaging, and loss of parts of the assembled device in the patient.

There is therefore a need for a catheter dome adapted to overcome the shortcomings of the prior art.

### SUMMARY OF THE INVENTION

The shortcomings of the prior art are generally mitigated by a catheter for transcutaneous and transluminal vacuum closure of perianal or entero-cutaneous fistulas and abscesses, the catheter comprising a body having a flexible and resilient enlarged portion, the body comprising a series of openings fluidly connected to a catheter chamber, and a tube configured to be connected to a vacuum generating system.

In another aspect of the invention, the catheter may comprise a resilient structure within the catheter chamber, wherein the resilient structure may be a spatial lattice structure allowing maintenance of the structural integrity of the catheter and promoting drainage, and wherein the enlarged portion and the resilient structure may be unitary. The body may further comprise ridges extending in a longitudinal direction of the enlarged portion, with the ridges being helicoidal, wherein the ridges may channel fluids, pus and tissue debris into the openings, and wherein the openings may be disposed between the ridges.

In yet another aspect of the invention, an outer surface of the catheter may be low friction and non-tissue adherent, and the enlarged portion may be made of biocompatible flexible and resilient material selected from silicone, PVC, and other medical grade polymers and elastomers. The catheter may further be made of material which is visible during medical imaging, the material of the catheter comprising a radio-opaque formulation or coating such as barium sulfate. The low friction characteristics of the outer surface of the catheter may enable withdrawal, advancement and replacement of the catheter without the necessity of general anesthesia.

In yet another aspect of the invention still, the catheter may comprise a closed internal channel for advancement of the catheter adapted to receive a rigid guidewire and a concentration of the openings may vary along a longitudinal axis of the body. An angle between an axis of an opening and a surface of the enlarged portion may also vary along a longitudinal axis of the enlarged portion, wherein the angle may be orthogonal at a lower portion of the enlarged portion and decreases near a tip of the enlarged portion. The catheter may be manufactured through a 3D printing process, wherein a size and geometry of the catheter may be custom fit to a patient's anatomy based on imaging data. The tube may comprise an angled portion and the enlarged portion may be shaped as a dome.

The present invention may also be realized by a kit comprising a plurality of the catheters, the catheters varying in size for the progressive closure of said fistulas and abscesses.

The present invention may further be realized by a method for treating perianal or entero-cutaneous fistula and abscess, the method comprising selecting a first catheter from a plurality of catheters having different lengths and diameters, each of the catheters comprising a body having a flexible and resilient enlarged portion which comprises a series of openings fluidly connected to a catheter chamber, inserting the first, catheter in the fistula or the abscess and, after a predetermined period of time, replacing the first catheter with a second catheter of the plurality of catheter, the enlarged portion of the second catheter having a smaller length and diameter than the enlarged portion of the first catheter.

In another aspect of the invention, the method may comprise characterizing the fistula or abscess and selecting one of the catheters based on the characterized fistula or abscess and inserting the first enlarged portion of the body may comprise rotatably inserting the first enlarged portion of the body into the fistula or the abscess.

The present invention may further yet be realized by a catheter for transcutaneous and transluminal vacuum closure of perianal or entero-cutaneous fistulas and abscesses, the catheter comprising a body comprising a series of openings fluidly connected to a catheter chamber, a tube in fluid communication with the body and connectable to a vacuum generating system, and a flange configured to define a maximum insertion length of the catheter into the fistula or abscess.

In another aspect of the invention, the catheter may comprise a flexible enlarged portion and a resilient structure within the enlarged portion, wherein the resilient structure may be a spatial lattice structure maintaining the, structural integrity of the catheter and promoting drainage, and wherein an outer surface of the body may comprise ridges extending in a longitudinal direction of the body, the ridges being helicoidal, and wherein the ridges may channel fluids, pus and tissue debris into the openings. An outer surface of the catheter may be low friction and non-tissue adherent.

Other and further aspects and advantages of the present invention will be obvious upon an understanding of the illustrative embodiments about to be described or will he indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is a front elevation view of a catheter in accordance with the principles of the present invention,

FIG. is a front close-up view of a catheter body of the catheter of FIG. 1.

FIG. 3 is a top plan view of the catheter of FIG. 1 or 2.

FIG. 4 is a bottom plan view of the catheter tube and body of FIG. 1.

FIG. 5A is a cross-sectional longitudinal view of the catheter body of FIG. 1 or 2.

FIG. 5B is a cross-sectional perspective bottom side view of the catheter body of FIG. 1 or 2.

FIG. 5C is a cross-sectional perspective top side view of the catheter body of FIG. 1 or 2.

FIG. 5D is a cross-sectional bottom plan view of the catheter body of FIG. 1 or 2

FIG. 6 is a front elevation view of an embodiment of a spatial lattice structure present in a catheter in accordance with the principles of the present invention.

FIG. 7 is a cross-sectional transverse view of the catheter of FIG. 1.

FIG. 8 is a left elevation view of another embodiment of a catheter halving an angular profile in accordance with the principles of the present invention.

FIG. 9 is a cross-sectional longitudinal view of the catheter of FIG. 8.

FIG. 10 is a side perspective view of yet another embodiment of a catheter in accordance with the principles of the present invention.

FIG. 11 is a top plan view of the catheter of FIG. 10.

FIG. 12 is a front elevation view of a plurality of catheters in accordance with the principles of the present invention.

FIG. 13 is a top plan vies of the catheter kit of FIG. 12.

FIG. 14 is a side elevation view of an embodiment of a catheter kit in accordance with the principles of the present invention.

FIG. 15 is a front view of the catheter of FIG. 8 fluidly connected to an active vacuum system.

FIG. 16 is a workflow diagram of a method of draining a fistula tract or abscess using the catheter kit of FIG. 14.

### DETAILED DESCRIPTION OF THE INVENTION

A novel dome catheter system for transcutaneous vacuum-assisted closure of perianal or enterocutaneous abscesses and fistula tracts and method thereof will be described hereinafter. Although the invention is described in terms of specific illustrative embodiments, it is to be understood that the embodiments described herein are by way of example only and that the scope of the invention is not intended to be limited thereby.

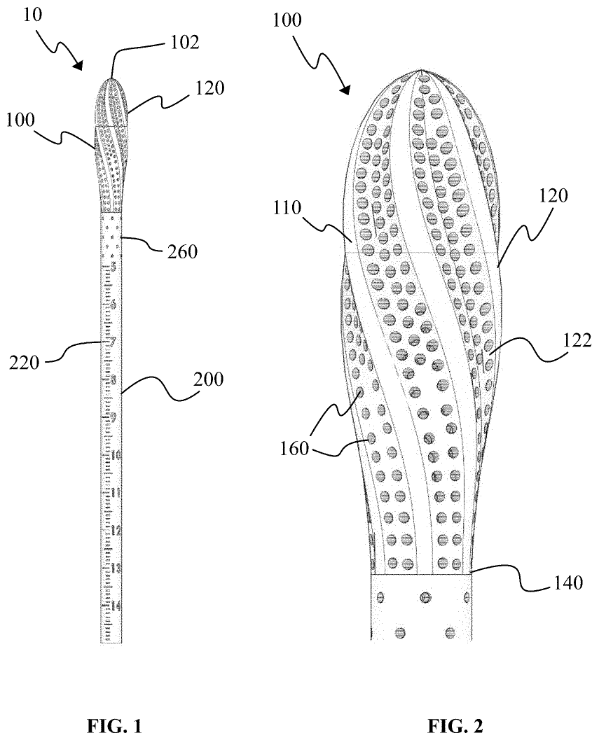

Referring to FIGS. 1 and 2, the present invention is broadly directed to a catheter 10 for transcutaneous vacuum closure of perianal or entero-cutaneous abscesses and fistulas. The catheter 10 generally comprises a catheter body 100 defining a domed tip 120. The domed tip 120 comprises an outer surface or periphery 122 having a plurality of openings or apertures 160. The catheter body 100 is substantially hollow, forming an inner catheter chamber 130. The openings 160 generally allows fluid access into the catheter chamber 130. The catheter body 100 may further comprise a lower portion 140 fluidly connected to a tube 200. The tube 200 may be connected to an active vacuum system such as, for example, a negative pressure pump or any other suitable system.

Still referring to FIGS. 1 and 2, in certain embodiments, the domed tip 120 defines a portion having a width being wider than the width of the tube 200 thereby allowing the catheter body 100 to occupy a greater volume within the abscess or fistula without enlarging the insertion orifice. The domed tip 120 may further encourage the growth of tissue within the abscess or fistula by ensuring contact of the catheter 10 with the internal surface of the abscess.

In preferred embodiments, the outer surface 122 of the catheter body 100 is smooth and free of irregularities. The smoothness of the outer surface 122 may allow easier insertion of the catheter 10 within an abscess or fistula while further generating less fluid friction with the pus and debris passing across the outer surface 122 and being vacuumed out of said abscess or fistula.

The catheter 10 may be made of a biocompatible flexible and resilient material, such as, but not limited to, silicone, PVC, and other medical grade polymers and elastomers. While the catheter 10 may be made of one material, in other embodiments, it may be desirable to produce other elements of the catheter using a different material. For example, the tube 200 may be made with one biocompatible material while the catheter body 100 of the catheter 10 may be made of another material. The resiliency of the catheter 10 may be selected to allow said catheter 10 to be easily inserted within an abscess or fistula while preventing said catheter 10 from collapsing once installed therein.

Still referring now to FIGS. 1 and 2, an embodiment of a catheter 10 is illustrated. In such embodiment, the outer surface 122 of the catheter body 100 generally comprises ridges 110 in the shape of a plurality of helicoid coils similar to a screw thread. As specified above, the openings or apertures 160 may be disposed between the ridges 110 and on a proximal portion of the tube 200. The positioning of the said apertures 160 generally allow fluid access to the inner chamber 130 of the catheter 10 and to the tube 200.

In a preferred embodiment, the ridges 110 longitudinally extend along a substantial portion or along the entirety of the catheter body 100. The ridges 110 are typically made of flexible yet resilient material. The length and the flexibility of the ridges 110 generally allow the catheter body 100 to collapse or having a reduced diameter to adapt to the size of a tract or abscess cavity in which the catheter body 100 is inserted. The length and flexibility further ease insertion and extraction of the catheter 10. Accordingly, the insertion or extraction of the catheter 10 may advantageously be performed by an individual lacking the qualifications of a surgeon such as, for example, by a specialized nurse in an outpatient clinic or even in a home care environment. Moreover, the insertion and/or extraction of the catheter 10 may be less painful for an individual thus allowing it, in certain cases, to be installed without the need of anesthesia,

The helical ridges 110 extending a substantial length of the catheter body 100 may further help channel fluids, namely pus and debris (such as granulation tissue), thereby allowing them to better flow through the catheter 10.

In certain embodiments, the outer surface of the catheter tube 200 may be completely smooth or be partially covered with apertures 260 for the purpose of drainage. In some embodiments, the tube 200 may further be covered with the measurement markings 220 designed to enable precise insertion.

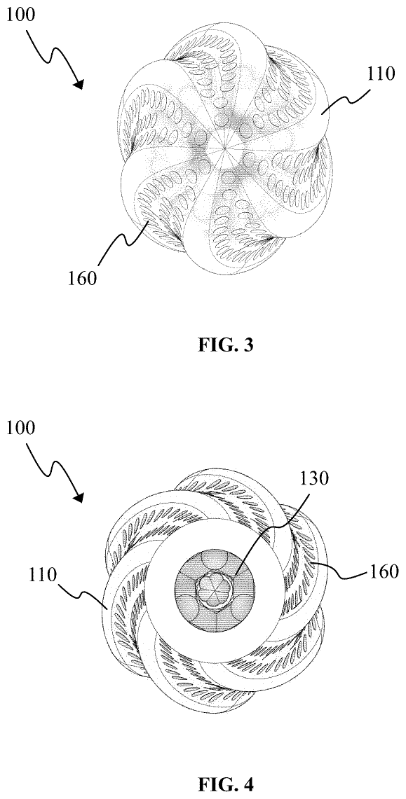

Referring now to FIGS. 3 to 5, the catheter head or body 100 is illustrated. As shown in FIG. 4, the inner chamber 130 of the body 100 comprises an inner resilient structure 170. The structure 170 generally maintains the shape of the catheter body 100 and further provides a resilient force when the body 100 is deformed, such as when inserted in a tract or fistula.

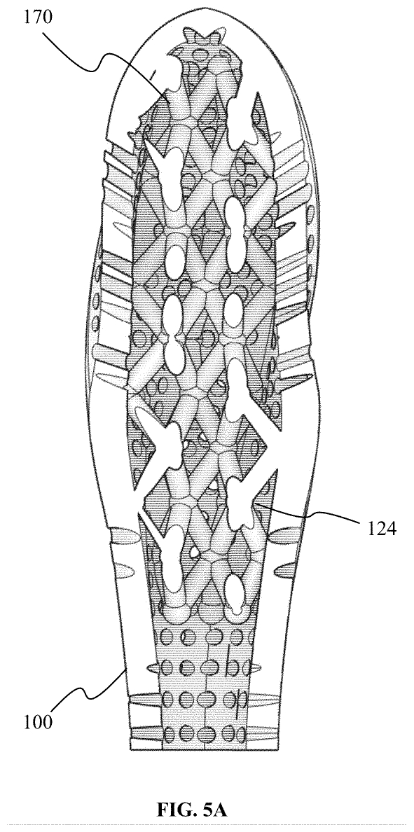

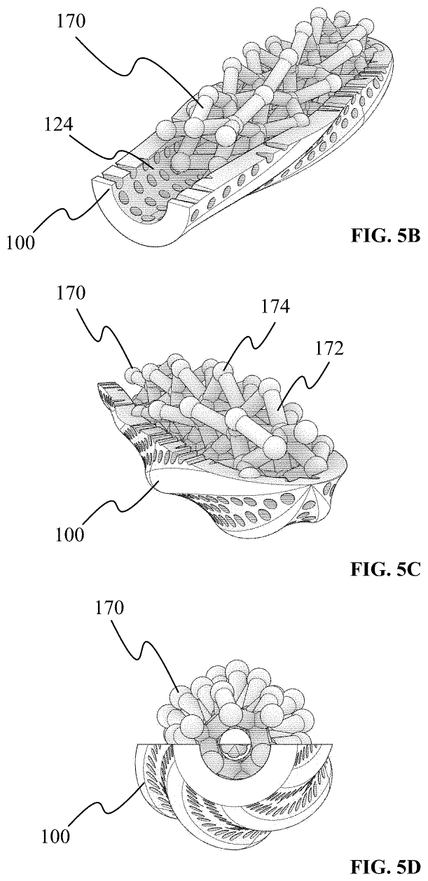

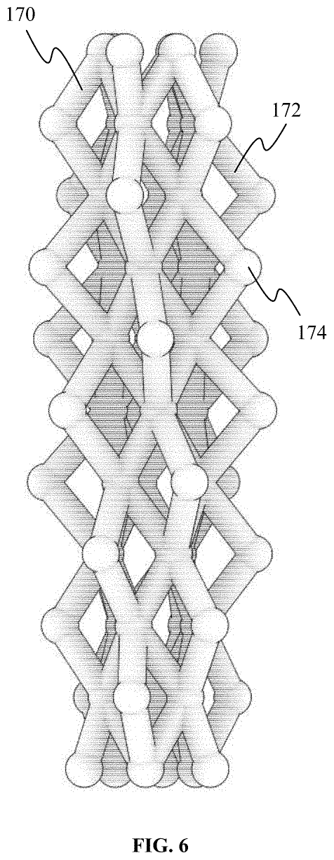

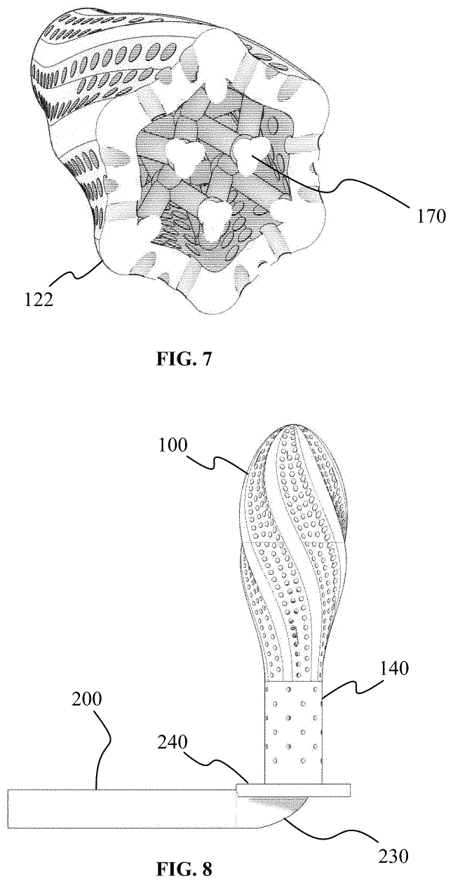

Referring now to FIGS. 5 to 7, in some embodiments, the resilient structure 170 may be a spatial lattice structure. The resilient structure generally aims at retaining a structural integrity of the catheter body 100 and at, preventing a collapse of the catheter body 100 in larger designs thereof. The structure 170 is typically disposed within the catheter chamber 130. The structure 170 may fill up the entirety or a part of the catheter chamber 130, depending on the size of the catheter body 100 and the desired proclivity to collapse.

In certain embodiments, the structure 170 may comprise a plurality of interconnected columns 172. The structure 170 may further comprise spherical knots 174 connected to the columns 172 or connected to an inner surface 124 of the catheter body 100. in further embodiments, the spherical knots 174 may contact the inner surface of the catheter body 100.

In preferred embodiments, the diameter of the columns 170 may range between 0.3 and 1.5 mm and the diameter of the spherical knots 174 may range between 0.3 and 1.5 mm. The length of the columns 172 may vary from 0.5 mm to 5 mm. Each of the spherical knots 174 may interconnect up to six columns 172. In the illustrated embodiment, the structure 170 comprises six sets of vertically disposed knots 174 at a same general position along the longitudinal axis of the catheter body 100, The columns 172 and knots 174 may he arranged in such a way to define canals within the internal lattice structure 170 along the longitudinal axis of the catheter body 100. In certain embodiments, the canals have an effective diameter of up to 4 mm thereby allowing the surgeon to insert a guidewire inside the catheter 10, if necessary.

The spatial lattice structure 170 of the columns 172 and knots 174 may be made of the same flexible and resilient biocompatible materials of the remainder of the catheter body 100. In certain embodiments, the spatial lattice structure 170 and the remainder of the catheter body 100 may be unitary and manufactured using a 3D minting technique. Understandably, different materials or arrangements of the spatial lattice structure 170 may be used in future embodiments, as long as the structure 170 supports the catheter body 100 from collapsing, while maintaining the flexibility of the said catheter 10.

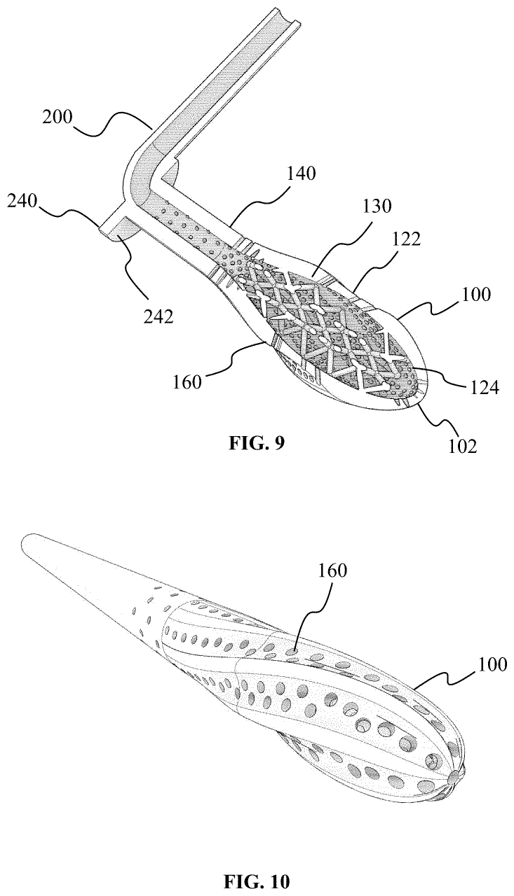

Referring now to FIGS. 8 and 9, an exemplary embodiment of the catheter 10 is illustrated having a catheter body 100 forming an angle with a longitudinal length of the tube 200. In the illustrated embodiment, the catheter body 100 is orthogonal to the tube 200, Understandably, the tube 200 may comprise a curved portion 230 defining an angle between the catheter body 100 and the tube 200. As an example, the tube may have a length of about 36 mm from the tip 102 of the catheter 10 to the constriction that is tangent to the tube. In such an example, the diameter of the catheter body 100 is, at its widest point, about 11 mm. On the other hand, the diameter of the catheter body 100 at the connection point with the tube is about 7.10 mm. At such a point, the diameter inside the mouth of the body chamber is about 4.05 mm. The wall of the catheter body is about 1.5 mm thick. The wall of the catheter body 100 is typically slightly thicker at the coils 110, and a bit thinner between the coils 110. Variable wall thickness generally allows the catheter to combine the rigidness with flexibility and patency.

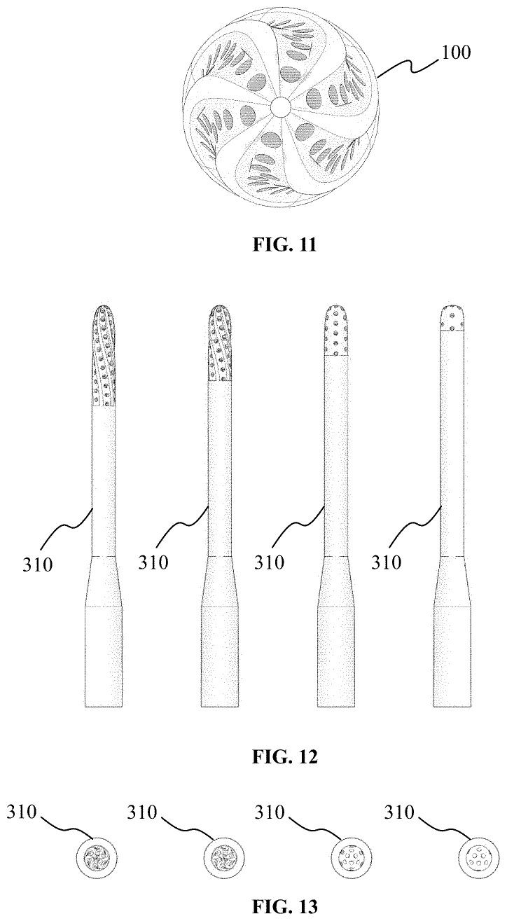

Referring now to FIGS. 9 to 11, different embodiments of the catheter body 100 are illustrated. The said catheter body 100 comprises apertures 160 having various shapes and sizes. In a preferred embodiment, the apertures 160 have an ellipsoidal or circular cross-section. The diameter of the apertures 160 may be in the range of 0.3 to 1.5 mm, preferably about 0.75 mm for circularly shaped apertures.

In certain embodiments, the apertures 160 may be oriented at an angle relative to the surface 122 of the catheter body 120. For example, and as shown in FIG. 9, a plurality of apertures 160′ may be angled relative to the surface 122. Certain apertures 160 may be angled such that apertures 160 near a tip 102 of the catheter body 100 are angled toward the tip, while the apertures 160 near the lower portion 140 are arranged normal or orthogonal to the surface 122 of the catheter body 100. The angling of the apertures 160 may vary gradually from the tip 102 to the lower portion 140 of the catheter body 100.

In a preferred embodiment, the apertures 160 are disposed between the ridges 110, as the ridges 110 provide structural support for the catheter body 100. Moreover, a denser arrangement of the apertures 160 on the surface 122 of the catheter body 100 may provide increased patency and prevent clogging. The denser arrangement generally implies having small apertures 160. In certain embodiments, the concentration of apertures 160 may vary along the longitudinal length of the catheter body 100. For example, in the illustrated embodiments, the concentration of apertures 160 varies gradually from a lowest density near the lower portion 140 and to a highest density near the tip 102. This arrangement of concentrations and the aforementioned angling of the apertures 160 may allow a greater pressure distribution inside the catheter chamber 130 with most of the pressure being exerted near the top of the chamber 130, while some pressure is still exerted near its lower parts. This may allow the top of the cavity to heal faster, while preventing the granulation tissue from overgrowing on the lower parts.

Broadly, the catheter 10 performs a more effective transcutaneous vacuum closure of perianal or entero-cutaneous abscesses and fistulas when it comprises a higher concentration of apertures 160 and apertures 160 having a smaller cross-sectional area. The number of apertures 160 or density shall provide enough flow to adequately drain the tissues around the inserted catheter 10.

Referring back to the embodiment illustrated at FIG. 8 and to the embodiment of FIG. 9, the catheter tube 200 may comprise an annular flange 240 facilitating better and easier sealing of the catheter with the skin around the wound exit when performing a transcutaneous insertion of the catheter 10. The flange 240 may allow the medical practitioner to insert the catheter 10 to a predefined depth without taking any measurements—with each catheter 10 in a treatment set (discussed further below) having a predefined height from the tip 102 of the catheter body 100 to the flange 240. Thus, the flange may advantageously control the depth of insertion of the catheter by acting as a stopper. In a preferred embodiment, the predefined height from the tip 102 of the catheter body 100 to the flange 240 is approximately 3 mm shorter than the depth of the abscess thereby creating an approximately 3 mm gap between the top portion 102 of the catheter 10 of the flange 240 and the deepest portion of the abscess. As such, the flange 240 creates a seal over the abscess aperture as the skin of the patient is in contact with a contact portion of the flange 240.

In some embodiments, by limiting the insertion depth of the catheter 10, the medical practitioner may easily select, the adequate catheter 10 head that has the specifications required for the specific situation (i.e., dimensions, shape, etc.).

As previously stated, the tube 200 may be angled behind the flange 240 with respect to the plane of the flange 240, which may facilitate a constant suction and protects the external tube 200 from bending, while providing the patient with a more comfortable placement of the tube 200. The flange 240 may have any suitable diameter or thickness. For example, the flange 240 may be about 3 mm thick or less with a diameter of about 20 mm. Moreover, the contact surface 242 may be adapted to contact the skin to ensure a desirable vacuum seal level. In certain embodiments, contact surface 242 may be configured to receive a stoma paste and may be flat, thus improving a better vacuum sealing level and/or ensuring a more secure installation of the catheter 10.

The external tube 200 may have any suitable internal and external diameters. For example, the tube 200 may have an external diameter of about 7 mm and an internal diameter of about 4 mm.

It may be appreciated that the catheter 10 may be made in any size and shape according to the requirements of a specific patient and can be custom 3D printed based on MR imaging of their septic cavity and tubing.

The present invention further comprises a parametric method of designing and manufacturing the catheter 10. In certain embodiments, the method may comprise measuring the septic cavity of a patient and inputting said measurements into a computerized system, such as using an interface. The method may further comprise the computerized system processing several measured parameters (e.g., height, depth, width and shape of the cavity) and automatically defining an adequate custom shape of one or more catheters designed for one particular patient's treatment.

In certain embodiments, a radio-opaque formulation may be used to produce the catheter 10 and/or a coating such as barium sulfate may be used to coat the catheter 10. A catheter 10 in accordance with these embodiments may be easier to locate using medical imaging means of the patient's body.

Referring now to FIGS. 12 and 13, a plurality of embodiments of catheter 310 in accordance with the principles of the present invention are illustrated. Such catheters 310 may have smooth surface 122 portions, such as not comprising ridges 110. In such embodiments, the catheter body 100 has different shapes and heights to adapt to different shapes and lengths of tracts and fistulas.

Referring now to FIG. 14, a catheter kit 300 comprising a plurality of catheters 310 having various dimensions is illustrated. As described in greater detail below, the catheters 310 may comprise catheter bodies 311 ranging from larger to smaller and which may be used throughout a complete treatment of a patient's septic cavity. For example, a large catheter 310A may be used early in the treatment of the patient and, as the patient's septic cavity heals and reduces in volume/size, progressively smaller catheters may be introduced until the smallest catheter 310B is used when little suction is required.

Still referring to FIG. 14, the catheter kit 300 comprises the plurality of catheters 310 having progressively smaller lengths and diameters and which may be used in sequence for the entire treatment, i.e., progressive closure of the septic cavity. As illustrated, the catheter 310 may comprise a flange 240. The catheter kit 300 may comprise a largest catheter 310A having dimensions associated with a patient's fistula or abscess size at the beginning of a treatment. The catheters 310 may thereafter be replaced or exchanged as necessary (for example, every 1 or 2 days) for progressively smaller and shorter catheters as the fistula or abscess heals and closes, If needed, the treatment may use, at last, the smallest catheter 310B. The exemplary catheter kit 300 illustrated in FIG. 14 comprises twenty-two catheters 310, each of the catheters 310 being 3 mm shorter than the previous one.

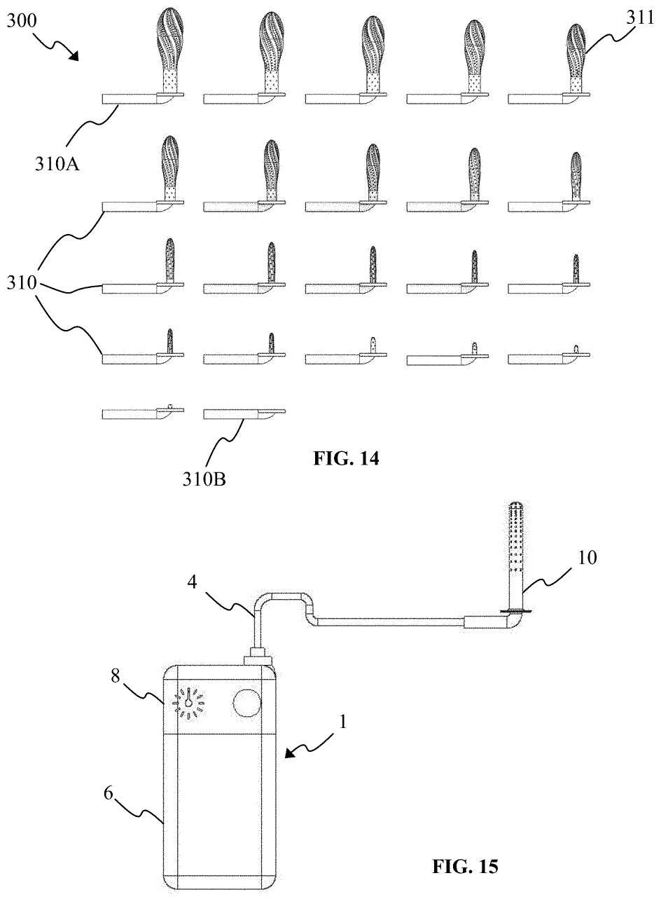

Referring now to FIG. 15, a catheter 10 connected to an active vacuum system or vacuum generating system 1 is illustrated, The system 1 generally comprises a tube 4, a pump 6 and a control unit 8 for controlling the pump 6. It may be appreciated that the tube 200 of the catheter 10 may be fluidly connected to the tube 4 using any connection means known in the art without departing from the invention, such as tube fittings. The pump 6 provides the catheter 10 with a suitable negative pressure for the vacuuming of pus and debris from the tract or abscess cavity. In certain embodiments, the negative pressure may be in a range from −25 mmHg to −125 mmHg, representing a standard in a negative pressure wound therapy. It may be appreciated that the desired negative pressure may vary according to the size of the catheter body 100. For example, lower values of negative pressure should be provided by the system 1 when used with a smaller catheter body 100. Medical practitioners may opt to use either lower or higher values of negative pressure without departing from the scope of the invention. In preferred embodiments, the entire vacuum generating system 1 is rendered airtight by using stoma paste and silicone dressing on flange 240 or tube 200 to attach the catheter 10 to the patient's skin.

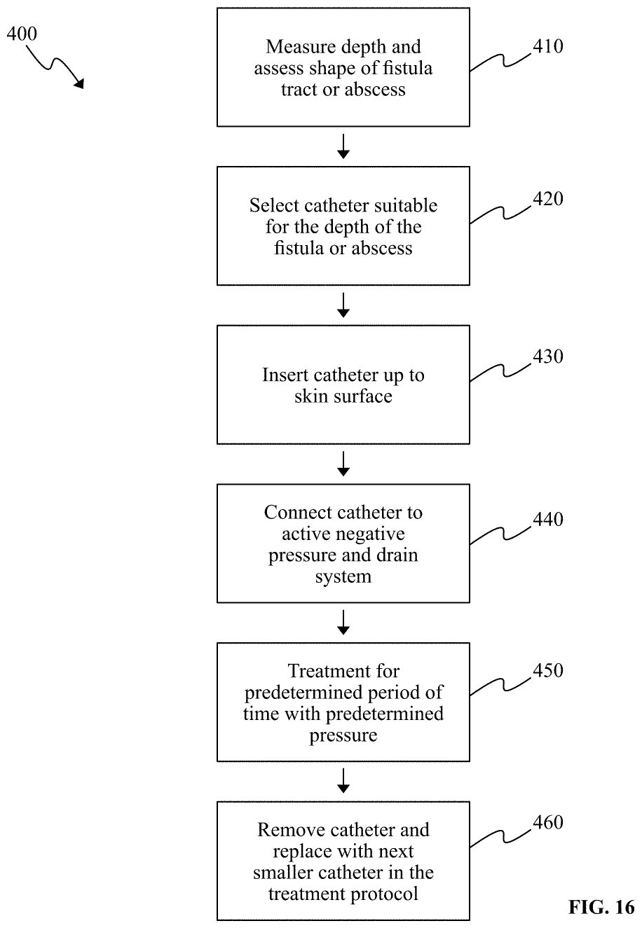

Referring now to FIG. 16, a method for using a system of catheters 310 for transcutaneous vacuum closure of perianal or entero-cutaneous fistulas and abscesses of progressively smaller length and diameters 400 is illustrated. The method 400 generally comprises measuring depth of a fistula tract or abscess 410. In some embodiments, the size of the cavity may be assessed or characterized. The method 400 further comprises selecting a first catheter 310A based on the assessed size of the cavity and suitable for its depth 420. In certain embodiments, the method 400 may further comprise leaving a 3 mm space or gap between the surface of the catheter 310 and walls of the cavity. In the case of a fistula, there may be a need to surgically close the internal opening. In such case it may be beneficial for the internal opening to heal before the catheter 310 is introduced. In all cases, the cavity should be cleaned before the procedure.

The method 400 further comprises inserting the sterile catheter 310 into the cavity 430. The insertion may be combined to a twist motion up to the skin surface. In embodiments where the catheters 310 comprise a flange, the flange may be inserted up to the patient's skin surface and the contact surface 242 of the flange may adhere to or at least contact the skin. In embodiments where the catheters 310 are straight, a careful measurement of the insertion is required for a good placement of the catheter 310.

In certain embodiments, inserting the catheter 430 may require using a guidewire inserted inside the catheter 310 which is removed once the catheter 310 is placed properly. Once the guidewire is removed, an end of the tube being opposite to the catheter body is connected to an active negative pressure and drain system 440. The negative pressure pump is typically capable of providing a suitable negative pressure. The connection may be performed via an additional external tube, In certain embodiments, one or more adapters may be used to connect the tube to the pump or to the external tube. In a preferred embodiment, a pump capable of generating a −125 mmHg negative pressure may be used and the pump may be mobile to allow the patient to function normally. The method 400 may further comprise adjusting the pump to generate a negative pressure in the range from −25 mmHg to −125 mmHg to allow for a more precise adjustment of the catheter's patency.

The method 400 further comprises performing the treatment for a predetermined period of time with a predetermined pressure 450. The pump should preferably remain airtight for the predetermined duration (typically 1-2 days) after which the cavity will have shrunk to the point that the catheter 310 needs to be exchanged for a smaller one. The method may therefore comprise the step of removing the catheter and replacing it with the next smaller catheter in the treatment protocol 460. Active drainage of the cavity may prevent accumulation of pus and debris and may allow healing i.e., shrinkage of the cavity.

The medical practitioner should make the decisions on how to carry out the treatment, for example, by prescribing additional painkillers or changing the pace of exchange of catheters, The MRI scans carried out throughout the treatment may additionally help a medical practitioner to assess how the treatment progresses. In future the treatment may be accompanied by a mobile application that will analyze patients' data and provide tips on how to carry out the treatment.

While illustrative and presently preferred embodiments of the invention have been described in detail hereinabove, it is to be understood that the inventive concepts may he otherwise variously embodied and employed and that the appended claims are intended to be construed to include such variations except insofar as limited by the prior art.

1. A catheter for transcutaneous and transluminal vacuum closure of perianal or entero-cutaneous fistulas and abscesses, the catheter comprising:
a body having a flexible and resilient enlarged portion, the body comprising a series of openings fluidly connected to a catheter chamber; and
a tube configured to be connected to a vacuum generating system.

2. The catheter of claim 1 further comprising a resilient structure within the catheter chamber.

3. The catheter of claim 2 where the resilient structure is a spatial lattice structure allowing maintenance of the structural integrity of the catheter and promoting drainage.

4. The catheter of claim 2, wherein the enlarged portion and the resilient structure are unitary.

5. The catheter of claim 1, wherein the body further comprises ridges extending in a longitudinal direction of the enlarged portion.

6. The catheter of claim 5, the ridges being helicoidal.

7. The catheter of claim 5, wherein the ridges channel fluids, pus and tissue debris into the openings.

8. The catheter of claim 5, wherein the openings are disposed between the ridges.

9. The catheter of claim 1, wherein an outer surface of the catheter is low friction and non-tissue adherent.

10. The catheter of claim 1, wherein the enlarged portion is made of biocompatible flexible and resilient material selected from silicone, PVC, and other medical grade polymers and elastomers.

11. The catheter of claim 1, Therein the catheter is made of material which is visible during medical imaging

12. The catheter of claim 11, the material of the catheter comprising a radio-opaque formulation or coating such as barium sulfate.

13. The catheter of claim 1. wherein the catheter comprises a dosed internal channel for advancement of the catheter adapted to receive a rigid guidewire.

14. The catheter of claim 1, wherein a concentration of the openings varies along a longitudinal axis of the body.

15. The catheter of claim 1, wherein an angle between an axis of an opening and a surface of the enlarged portion varies along a longitudinal axis of the enlarged portion.

16. The catheter of claim 15, wherein the angle is orthogonal at a lower portion of the enlarged portion and decreases near a tip of the enlarged portion.

17. The catheter of claim 1, wherein the catheter is manufactured through a 3D printing process.

18. The catheter of claim 17, wherein a size and geometry of the catheter is custom fit to a patient's anatomy based on imaging data.

19. The catheter of claim 1, wherein the tube comprises an angled portion.

20. The catheter of claim 1, the enlarged portion being shaped as a dome.

21. A kit comprising a plurality of the catheters of claim 1, the catheters varying in size for the progressive closure of said fistulas and abscesses.

22. A method for treating perianal or entero-cutaneous fistula and abscess, the method comprising:
selecting a first catheter from a plurality of catheters having different lengths and diameters, each of the catheters comprising a body having a flexible and resilient enlarged portion which comprises a series of openings fluidly connected to a catheter chamber;
inserting the first catheter in the fistula or the abscess;
replacing the first catheter with a second catheter of the plurality of catheter, the enlarged portion of the second catheter having a smaller length and diameter than the enlarged portion of the first catheter.

23. The method of claim 22, the method further comprising characterizing the fistula or abscess and selecting one of the catheters based on the characterized fistula or abscess.

24. The method of claim 2 wherein inserting the first enlarged portion of the body comprises rotatably inserting the first enlarged portion of the body into the fistula or the abscess.

25. A catheter for transcutaneous and transluminal vacuum closure of perianal or entero-cutaneous fistulas and abscesses, the catheter comprising:
a body comprising a series of openings fluidly connected to a catheter chamber;
a tube in fluid communication with the body and connectable to a vacuum generating system; and
a flange configured to define a maximum insertion length of the catheter into the fistula or abscess.

26 The catheter of claim 25 further comprising a flexible enlarged portion and a resilient structure within the enlarged portion.

27. The catheter of claim 26 wherein the resilient structure is a spatial lattice structure maintaining the structural integrity of the catheter and promoting drainage.

28. The catheter of claim 25, wherein an outer surface of the body comprises ridges extending in a longitudinal direction of the body.

29. The catheter of claim 28, the ridges being helicoidal.

30. The catheter of claim 28, wherein the ridges channel fluids, pus and tissue debris into the openings.

31. The catheter of claim 25, wherein an outer surface of the catheter is low friction and non-tissue adherent.
